(19) United States
(12) Patent Application Publication
(10) Pub. No.: US 2024/0321460 A1
(43) Pub. Date: Sep. 26, 2024

(54) METHODS AND SYSTEMS FOR ALERTING USERS TO HEALTH EMERGENCIES

(71) Applicant: ECHAS LLC, Auburndale, MA (US)

(72) Inventors: Amar Dhand, Brookline, MA (US); James E. Muller, Auburndale, MA (US); Anubodh S. Varshney, Boston, MA (US); Joseph Yoon, Brookline, MA (US); Josko Silobrcic, Brookline, MA (US)

(21) Appl. No.: 18/573,663

(22) PCT Filed: Jun. 29, 2022
(86) PCT No.: PCT/US22/35479

§ 371 Date: Dec. 22, 2023

Related U.S. Application Data
(60) Provisional application No. 63/216,512, filed on Jun. 29, 2021.

(51) Int. Cl. G16H 50/30; G08B 25/01; G16H 10/60

(52) U.S. Cl. CPC G16H 50/30; G08B 25/016; G16H 10/60

(57) ABSTRACT

The present disclosure provides methods and systems for alerting users to a likelihood that they are experiencing a health emergency. A method may include comparing current health data for a user to patient history data for the user to determine one or more probabilistic measurements corresponding to a likelihood that a user is undergoing a health emergency, such as a cardiac emergency and/or neurological emergency. If the probabilistic measurement(s) exceed a threshold, the method may include outputting a notification, such as an auditory and/or graphical notification, automatically alerting (e.g., calling) emergency medical services, or both. The method may be initiated by a user at the onset of one or more symptoms by opening an application or webpage. The method may use this initial input of symptom(s) to determine whether to proceed with requesting, and receiving, current health data to assist in the determination of the one or more probabilistic measurements.

### PRIORITY APPLICATION

This application claims the benefit of U.S. Provisional Patent Application No. 63/216,512, filed on Jun. 29, 2021, the disclosure of which is hereby incorporated by reference herein in its entirety.

### TECHNICAL FIELD

This disclosure relates generally to methods and systems for assisting users in determining whether they are experiencing a medical emergency.

### BACKGROUND

Many medical emergencies are experienced outside of medical settings. That is, medical emergencies, such as myocardial infarction or stroke, commonly occur to persons in their everyday lives, outside of hospitals or clinics. Many people do not seek emergency medical assistance when undergoing such events for a variety of reasons, including that they are not sure of the severity of the situation or are panicked by one or more symptoms, even though emergency medical services are generally associated with improved health outcomes. Additionally, some patients who have experienced prior cardiac and/or neurological emergencies are less prone to seeking assistance of an emergency medical service when they undergo a subsequent cardiac and/or neurological emergency. The inability of people to accurately assess their current health status during potential medical emergencies can cause both false positives and false negatives in seeking emergency medical assistance.

### SUMMARY

The present disclosure provides, inter alia, methods and systems for providing additional information regarding their current health status that can assist in alerting a person to a likelihood of a health emergency for the person. Health emergencies can be highly time sensitive and people may be unsure or unwilling to seek assistance from an emergency medical service based on their own qualitative assessment of their current health status. For example, a person may feel unwell but not to a degree where (s)he feels compelled to seek assistance. The methods and systems disclosed herein can provide more detailed information, or a summary (e.g., likelihood) derived from more detailed information, to a person about his/her current health status to alert to a possibility that (s)he is experiencing a health emergency. In some embodiments, current health data is compared to patient history data to determine one or more probabilistic measurements that correspond to a likelihood a person is experiencing a health emergency, such as a cardiac emergency and/or neurological emergency). This is an improvement over available alternatives, such as symptom checking apps, that can only at best allow a user to enter self-description of symptom(s) and output possible medical diagnoses that may correspond to those symptom(s). Methods and systems disclosed herein may be tailored to individual users at least because they may perform comparisons that account for a user's medical history in making any assessment of likelihood that a health emergency is occurring (e.g., by one or more probabilistic measurements). Moreover, the specific combination of different types of information, such as patient history, currently observed symptoms, current health data (e.g., biometric data, such as electrocardiogram data, facial and/or vocal recognition data, finger tapping), and one or more triggers, assessed quickly (e.g., without seeking and obtaining input of a physician or other medical profession first) can lead to determining one or more probabilistic measurements that more accurately reflect the likelihood that a user is experiencing a cardiac emergency and/or neurological emergency and therefore increase the likelihood that the user receives proper treatment, for example by quickly notifying a user to seek emergency medical services and/or alerting emergency medical services to the user's condition. By combining different types of information together when determining one or more probabilistic measurements for a user possibly experiencing a cardiac emergency and/or neurological emergency, false positives and false negatives can be simultaneously reduced.

In some embodiments, a method for alerting a user to a likelihood of a health emergency for the user includes receiving, by a processor of a computing device (e.g., a mobile phone, smart watch, tablet, or personal computer), patient history data for the user; receiving, by the processor, current health data from the user; and determining, by the processor, one or more probabilistic measurements at least in part by comparing the current health data to the patient history data, the one or more probabilistic measurements corresponding to the likelihood of a health emergency (e.g., a cardiac emergency, e.g., heart attack, and/or neurological emergency, e.g., stroke).

In some embodiments, the method comprises determining, by the processor, that at least a portion of the one or more probabilistic measurements exceed a threshold. Subsequently, a notification (e.g., a graphical notification, an auditory notification, or both) may be output, by the processor, to the user (e.g., wherein the notification indicates a value of the at least a portion of the one or more probabilistic measurements) (e.g., wherein the notification indicates a likelihood of a health emergency and/or instructs the user to contact an emergency service provider). Subsequently, the processor may automatically notify (e.g., calling) a remote emergency service provider (e.g., an ambulance service) to inform the provider of a medical emergency for the user (e.g., automatically calling 911). In some embodiments, the method comprises transmitting, by the processor, to the remote emergency service provider, at least a portion of the current health data and/or at least a portion of the one or more probabilistic measurements.

In some embodiments, receiving the patient history data comprises receiving, by the processor, at least a portion of the patient history data from input provided by the user (e.g., into a graphical user interface). In some embodiments, receiving the patient history data comprises receiving, by the processor, at least a portion of the patient history data from an electronic medical records database.

In some embodiments, receiving the current health data comprises receiving, by the processor, input data from one or more sensors (e.g., integrated into one or more wearables and/or one or more smart patches) in communication with the processor. In some embodiments, receiving the current health data comprises receiving, by the processor, at least a portion of the current health data from input manually provided by the user (e.g., into a graphical user interface).

In some embodiments, the patient history data and the current health data both comprise data corresponding to one or more vital signs, neurological status, an electrocardiogram, or one or more other cardiovascular parameters.

In some embodiments, the method comprises determining, by the processor, that one or more probabilistic measurements have not been made within a period of time. The method may further comprise requesting, with a graphical user interface, by the processor, that the user input additional current health data. In some embodiments, the method comprises receiving, by the processor, the additional current health data (e.g., corresponding to one or more vital signs, neurological status, an electrocardiogram, or one or more other cardiovascular parameters) from one or more sensors (e.g., integrated into one or more wearables and/or one or more smart patches) in communication with the processor and/or input manually provided by the user (e.g., into a graphical user interface).

In some embodiments, the method comprises receiving, by the processor, input from the user of one or more symptoms of concern. The method may further comprise determining, by the processor, the one or more symptoms are indicative of a potential health emergency. The method may further comprise requesting, by the processor, (e.g., by a graphical user interface) the current health data based on the input corresponding to the one or more symptoms of concern. In some embodiments, requesting the current health data comprises providing, by the processor, a notification to the user to request the user use one or more sensors to acquire at least a portion of the current health data.

In some embodiments, the health emergency is a cardiac event (e.g., heart attack) and/or neurological event (e.g., stroke), and/or other related medical condition (e.g., pulmonary embolism).

In some embodiments, the patient history data comprise one or more of intensity, duration, location, and pattern of one or more prior cardiac symptoms (e.g., chest pain) or baselines and the current health data comprise one or more of intensity, duration, and location of one or more current cardiac symptoms. Comparing the current health data to the patient history data may comprise comparing intensity of the one or more current cardiac symptoms to intensity of the one or more prior cardiac symptoms or baselines. Comparing the current health data to the patient history data may comprise comparing duration of the one or more current cardiac symptoms to duration of the one or more prior cardiac symptoms or baselines. Comparing the current health data to the patient history data may comprise comparing location of the one or more current cardiac symptoms to location of the one or more prior cardiac symptoms or baselines. Comparing the current health data to the patient history data may comprise comparing pattern of the one or more current cardiac symptoms to pattern of the one or more prior cardiac symptoms or baselines. Comparing the current health data to the patient history data may comprise a combination of the foregoing comparisons.

In some embodiments, the method comprises receiving, by the processor, input from the user of one or more symptoms of concern. The method may further comprise matching, by the processor, the one or more symptoms of concern to one or more symptoms of a cardiac emergency. The method may further comprise requesting, by the processor, (e.g., by a graphical user interface) the current health data based on the matching.

In some embodiments, the method comprises determining, by the processor, that the current health data corresponds to a cardiac health status no better than a cardiac health status that the patient history data corresponds to. In response, the processor may alter one or more of the one or more probabilistic measurements (e.g., incrementing the one or more of the one or more probabilistic measurements, e.g., by increasing a value of (e.g., adding points to) one or more of the one or more probabilistic measures).

In some embodiments, the patient history data comprise one or more baseline cardiac physical signs (e.g., blood pressure, sweat (e.g., rate and/or composition thereof)) and the current health data comprise one or more current cardiac physical signs (e.g., blood pressure, sweat (e.g., rate and/or composition thereof)) (e.g., received from one or more sensors in communication with the processor). Comparing the current health data to the patient history data may comprise comparing the one or more current cardiac physical signs to the one or more baseline cardiac physical signs. In some embodiments, the method comprises receiving, by the processor, the one or more current physical signs from one or more sensors in communication with the processor.

In some embodiments, comparing the one or more current cardiac physical signs to the one or more cardiac baseline physical signs comprises determining, by the processor, a presence of (i) a new abnormality in the current health data, (ii) a persistent abnormal physical sign in the current health data, or (iii) both (i) and (ii). The method may comprise, in response to determining the presence of (i), (ii), or (iii), altering, by the processor, one or more of the one or more probabilistic measurements (e.g., incrementing the one or more of the one or more probabilistic measurements, e.g., by increasing a value of (e.g., adding points to) one or more of the one or more probabilistic measures). In some embodiments, the method comprises determining, by the processor, the presence of (ii) or (iii) and, in response, requesting, by the processor, additional current health data comprising one or more of the one or more current cardiac physical signs.

In some embodiments, the current health data comprise at least a portion of a current electrocardiogram (e.g., received from one or more sensors in communication with the processor) and the patient history data comprise at least a portion of a baseline electrocardiogram. Comparing the current health data to the patient history data may comprise comparing the at least a portion of the current electrocardiogram to the at least a portion of the baseline electrocardiogram. In some embodiments, the method comprises determining, by the processor, that the at least a portion of the current electrocardiogram compared to the at least a portion of the baseline electrocardiogram matches a pattern for a cardiac emergency (e.g., ST segment elevation in at least two contiguous leads). In response, the processor may alter one or more of the one or more probabilistic measurements (e.g., incrementing the one or more of the one or more probabilistic measurements, e.g., by increasing a value of (e.g., adding points to) one or more of the one or more probabilistic measures).

In some embodiments, determining the one or more probabilistic measurements comprises totaling one or more scores indicative of a cardiac emergency based on comparing one or more cardiac symptoms, one or more physical signs, an electrocardiogram, or combinations thereof in the current health data to corresponding one(s) in the patient history data. In some embodiments, the method comprises determining, by the processor, that the one or more total scores exceed one or more thresholds and automatically notifying, by the processor, the user (e.g., graphically and/or audibly) and/or a remote emergency service provider.

In some embodiments, the patient history data comprise one or more of intensity, duration, and location of one or more prior neurological symptoms (e.g., slurred speech, balance problems, right-sided weakness) or baselines and the current health data comprise one or more of intensity, duration, and location of one or more current neurological symptoms. Comparing the current health data to the patient history data may comprise comparing intensity of the one or more current neurological symptoms to intensity of the one or more prior neurological symptoms or baselines. Comparing the current health data to the patient history data may comprise comparing duration of the one or more current neurological symptoms to duration of the one or more prior neurological symptoms or baselines. Comparing the current health data to the patient history data may comprise comparing location of the one or more current neurological symptoms to location of the one or more prior neurological symptoms or baselines. Comparing the current health data to the patient history data may comprise a combination of the forgoing comparisons.

In some embodiments, the method comprises receiving, by the processor, input from the user of one or more symptoms of concern. The method may further comprise matching, by the processor, the one or more symptoms of concern to one or more symptoms of a neurological emergency. The method may further comprise requesting, by the processor, (e.g., by a graphical user interface) the current health data based on the matching.

In some embodiments, the method comprises determining, by the processor, that the current health data corresponds to a neurological health status no better than a neurological health status that the patient history data corresponds to. The processor may, in response, alter one or more of the one or more probabilistic measurements (e.g., incrementing the one or more of the one or more probabilistic measurements, e.g., by increasing a value of (e.g., adding points to) one or more of the one or more probabilistic measures).

In some embodiments, the patient history data comprise one or more baseline neurological physical signs (e.g., blood pressure, sweat (e.g., rate and/or composition thereof)) and the current health data comprise one or more current neurological physical signs (e.g., blood pressure, sweat (e.g., rate and/or composition thereof)) (e.g., received from one or more sensors in communication with the processor). Comparing the current health data to the patient history data may comprise comparing the one or more current neurological physical signs to the one or more baseline neurological physical signs. In some embodiments, the method comprises receiving, by the processor, the one or more current neurological physical signs from one or more sensors in communication with the processor.

In some embodiments, comparing the one or more current neurological physical signs to the one or more baseline neurological physical signs comprises determining, by the processor, a presence of (i) a new abnormality in the current health data, (ii) a persistent abnormal physical sign in the current health data, or (iii) both (i) and (ii). The method may comprise, in response to determining the presence of (i), (ii), or (iii), altering, by the processor, one or more of the one or more probabilistic measurements (e.g., incrementing the one or more of the one or more probabilistic measurements, e.g., by increasing a value of (e.g., adding points to) one or more of the one or more probabilistic measures). In some embodiments, the method comprises determining, by the processor, the presence of (ii) or (iii) and, in response, requesting, by the processor, additional current health data comprising one or more of the one or more current neurological physical signs.

In some embodiments, the current health data comprise at least a portion of a current electrocardiogram (e.g., received from one or more sensors in communication with the processor) and the patient history data comprise at least a portion of a baseline electrocardiogram. Comparing the current health data to the patient history data may comprise comparing the at least a portion of the current electrocardiogram to the at least a portion of the baseline electrocardiogram. In some embodiments, the method comprises determining, by the processor, that the at least a portion of the current electrocardiogram comprises a new arrhythmia compared to the at least a portion of the baseline electrocardiogram. In some embodiments, the processor may, in response, alter one or more of the one or more probabilistic measurements (e.g., incrementing the one or more of the one or more probabilistic measurements, e.g., by increasing a value of (e.g., adding points to) one or more of the one or more probabilistic measures).

In some embodiments, the patient history data comprise a current neurological exam and the current health data comprise a baseline neurological exam. Comparing the current health data to the patient history data may comprise comparing the current neurological exam to the baseline neurological exam.

In some embodiments, comparing the current neurological exam to the baseline neurological exam comprises determining, by the processor, the current neurological exam comprises a change from the baseline neurological exam that matches a neurological emergency (e.g., new language difficulty and right-sided weakness). The method may further comprise, in response to determining the change that matches the neurological emergency, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

In some embodiments, determining the one or more probabilistic measurements comprises totaling one or more scores indicative of a neurological emergency based on comparing one or more neurological symptoms, one or more physical signs, an electrocardiogram, or combinations thereof in the current health data to corresponding one(s) in the patient history data.

The method may comprise determining, by the processor, that the one or more total scores exceed one or more thresholds; and automatically notifying, by the processor, the user (e.g., graphically and/or audibly) and/or a remote emergency service provider.

The method may comprise determining, by the processor, the one or more probabilistic measurements at least in part by matching the current health data to one or more independent baseline criteria. In some embodiments, the one or more independent baseline criteria comprise one or more high-risk symptoms, one or more high-risk physical signs, or a high-risk electrocardiogram. In some embodiments, the method comprises altering, by the processor, the one or more probabilistic measurements based on the matching to the one or more independent baseline criteria independent of the patient history data.

In some embodiments, the one or more probabilistic measurements are determined using predetermined risk scores for health emergencies (e.g., cardiac and/or neurological risk scores).

In some embodiments, the patient history data comprise one or more baseline physical signs (e.g., blood pressure, sweat (e.g., rate and/or composition thereof)) and the current health data comprise one or more current physical signs (e.g., blood pressure, sweat (e.g., rate and/or composition thereof)) (e.g., received from one or more sensors in communication with the processor). Comparing the current health data to the patient history data may comprise comparing the one or more current physical signs to the one or more baseline physical signs.

In some embodiments, the method comprises receiving, by the processor, the one or more current physical signs from one or more sensors in communication with the processor.

In some embodiments, the method comprises receiving, by the processor, input from the user of one or more symptoms of concern. The method may further comprise determining, by the processor, that the current health data (and optionally the patient history data) is incomplete for the one or more symptoms of concern. The method may further comprise, upon determining that the current health data is incomplete, requesting, by the processor, (e.g., by a graphical user interface) additional current health data.

In some embodiments, the method comprises receiving, by the processor, the additional current health data, wherein the additional current health data corresponds to a different type of measurement than current health data.

In some embodiments, the additional current health data is from one or more second sensors and the current health data is from one or more first sensors that are different from the one or more second sensors (e.g., in one or more different wearables and/or one or more different smart patches). In some embodiments, the additional current health data is received from one or more smart patches and the current health data or is received from one or more wearables or wherein the additional current health data is received from one or more wearables and the current health data or is received from one or more smart patches.

In some embodiments, comparing the one or more current physical signs to the one or more baseline physical signs comprises determining, by the processor, a presence of (i) a new abnormality in the current health data, (ii) a persistent abnormal physical sign in the current health data, or (iii) both (i) and (ii). The method may further comprise, in response to determining the presence of (i), (ii), or (iii), altering, by the processor, one or more of the one or more probabilistic measurements (e.g., incrementing the one or more of the one or more probabilistic measurements, e.g. by increasing a value of (e.g., adding points to) one or more of the one or more probabilistic measures).

In some aspects, a system comprises a processor and a memory, the memory having instructions stored thereon that, when executed by the processor, cause the processor to perform a method as disclosed herein. In some embodiments, the processor and the memory are comprised in a mobile phone, smart watch, tablet, or personal computer.

A smart first aid kit for acquiring health data from a user when the user may be undergoing a health emergency may comprise one or more wearables sized and shaped to be worn by the user and operable to acquire first health data and transmit (e.g., wirelessly) the first health data to a computing device (e.g., a mobile phone, a smart watch, a tablet, or a personal computer). A smart first aid kit for acquiring health data from a user when the user may be undergoing a health emergency may comprise (e.g., alternatively or additionally) one or more smart patches sized and shaped to be temporarily adhered to skin of the user and operable to acquire second health data and transmit (e.g., wirelessly) the second health data to a computing device (e.g., mobile phone, smart watch, tablet, or personal computer).

In some embodiments, the one or more wearables comprises a wrist band, a shirt (e.g., with integrated electrodes), or both. In some embodiments, the one or more wearables comprises the wrist band and the wrist band comprises a blood pressure module, a pulse module, an oxygen saturation module, a sweat monitor module (e.g., a sweat rate monitor module), or a combination thereof.

In some embodiments, the one or more smart patches comprises two wrist patches, an ankle patch, and a chest (e.g., nipple) patch. In some embodiments, the one or more smart patches comprises a plurality of smart patches that are together operable to acquire electrocardiogram data or sweat presence data. In some embodiments, (i) the one or more smart patches are operable to analyze ST segment(s), cardiac rhythm, or pulse variability from the electrocardiogram data or sweat presence from the sweat presence data or (ii) the one or more smart patches are operable to transmit data (e.g., wirelessly) to the one or more wearables and the one or more wearable are operable to analyze ST segment(s), cardiac rhythm, or pulse variability from the electrocardiogram data or sweat presence from the sweat presence data.

In some aspects, a method for acquiring health data from a user when the user may be undergoing a health emergency may comprise providing, by a processor of a computing device (e.g., a mobile phone, a smart watch, a tablet, or a personal computer), a notification (e.g., graphically or audibly) to acquire health data using one or more smart patches; and receiving, by the processor, health data from a plurality of smart patches that have been placed at both wrists, the left ankle, and the left nipple of the user, wherein the health data is electrocardiogram data or sweat presence data.

Any two or more of the features described in this specification, including in this summary section, may be combined to form implementations not specifically explicitly described in this specification.

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system comprised of one or more processing devices and memory storing instructions that are executable by the one or more processing devices to perform various control operations.

### BRIEF DESCRIPTION OF THE DRAWINGS

Drawings are presented herein for illustration purposes, not for limitation. The foregoing and other objects, aspects, features, and advantages of the disclosure will become more apparent and may be better understood by referring to the following description taken in conjunction with the accompanying drawings, in which:

FIG. 1A is a flow diagram of a method for alerting a user to a likelihood of a health emergency for the user, according to illustrative embodiments of the present disclosure;

FIG. 1B is a flow diagram of a method for comparing current health data to patient history data that can be used in the method of FIG. 1A, according to illustrative embodiments of the present disclosure;

FIG. 1C is a flow diagram of a method for determining one or more probabilistic measurements corresponding to the likelihood of a health emergency that can be used in the method of FIG. 1A, according to illustrative embodiments of the present disclosure;

FIG. 2 is a flow diagram of a method for using a smart first aid kit to alert a user to a likelihood of a health emergency for the user, according to illustrative embodiments of the present disclosure;

FIG. 3 is a block diagram of an example network environment for use in the methods and systems described herein, according to illustrative embodiments of the present disclosure; and

FIG. 4 is a block diagram of an example computing device and an example mobile computing device, according to illustrative embodiments of the present disclosure.

### DETAILED DESCRIPTION OF CERTAIN EMBODIMENTS

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems according to certain embodiments of the present disclosure that consist essentially of, or consist of, the recited components, and that there are processes and methods according to certain embodiments of the present disclosure that consist essentially of, or consist of, the recited processing steps.

In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; (iv) the terms “about” and “approximately” may be understood to permit standard variation as would be understood by those of ordinary skill in the relevant art; and (v) where ranges are provided, endpoints are included. It should be understood that the order of steps or order for performing certain action is immaterial so long as operability is not lost. Moreover, two or more steps or actions may be conducted simultaneously. Headers are provided for the convenience of the reader and are not intended to be limiting with respect to the claimed subject matter. As used herein, unless otherwise clear from context, the terms “patient,” “person,” and “user” are used interchangeably and the terms “alerting” and “notifying” are used interchangeably.

The present disclosure provides, inter alia, methods and systems for alerting users to a likelihood that they are experiencing a health emergency. The methods and systems may be implemented using personal computing devices, such as, for example mobile phones, tablets, laptops, or smart watches. A method may include comparing current health data for a user to patient history data (e.g., received from electronic medical records for the user) for the user to determine one or more probabilistic measurements corresponding to a likelihood that a user is undergoing a health emergency, such as a cardiac emergency (e.g., heart attack, myocardial infarction) and/or neurological emergency (e.g., stroke). If the probabilistic measurement(s) exceed a threshold, the method may include outputting a notification, such as an auditory and/or graphical notification to the user, automatically alerting (e.g., calling) emergency medical services, or both. The user may initiate the method at the onset of one or more symptoms by opening an application or webpage to enter the one or more symptoms. The method may use this initial input of symptom(s) to determine whether to proceed with requesting, and receiving, current health data to assist in the determination of the one or more probabilistic measurements. In some embodiments, a probabilistic measurement is made and/or a user is notified and/or emergency medical services are called based on current health data even without comparison to patient history data for the user.

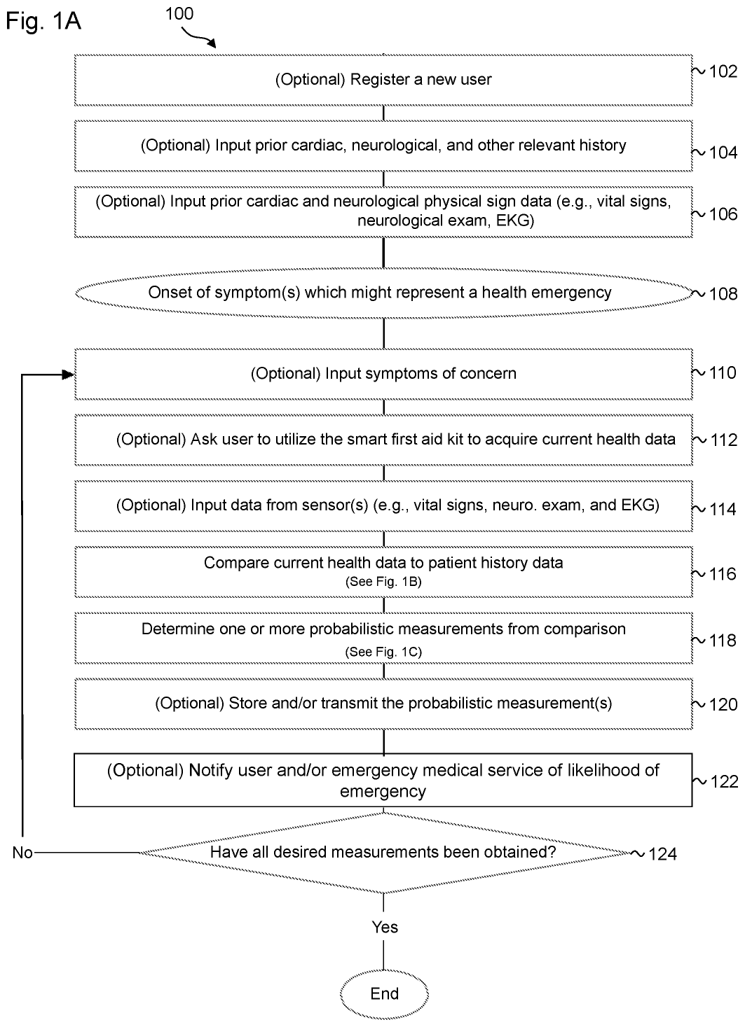

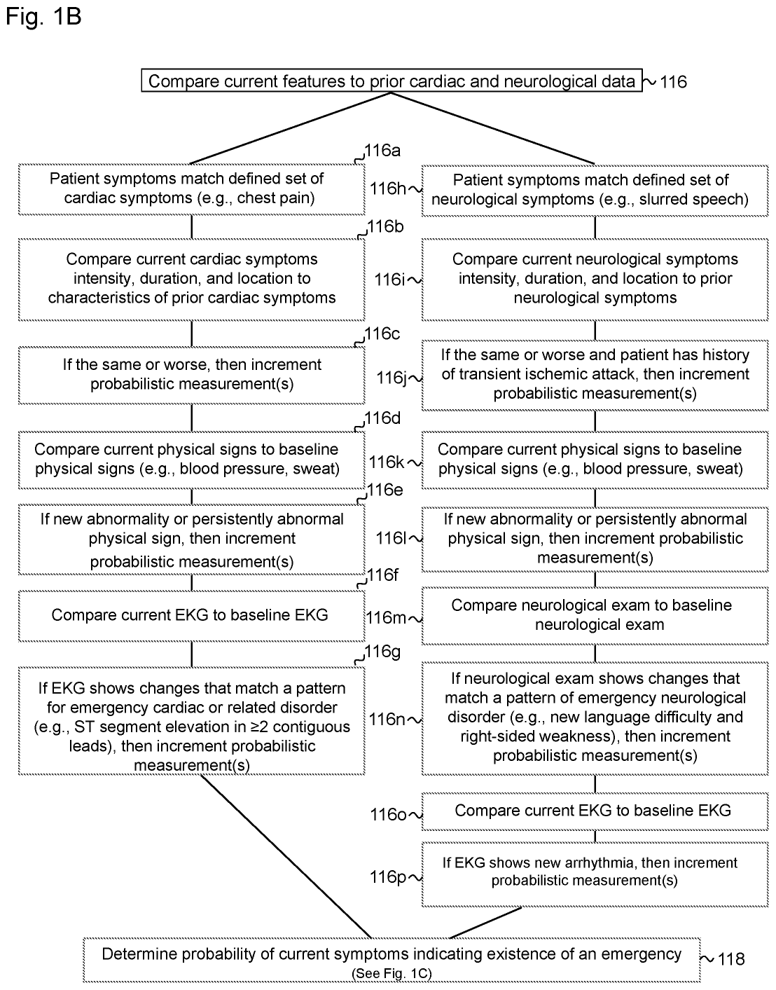

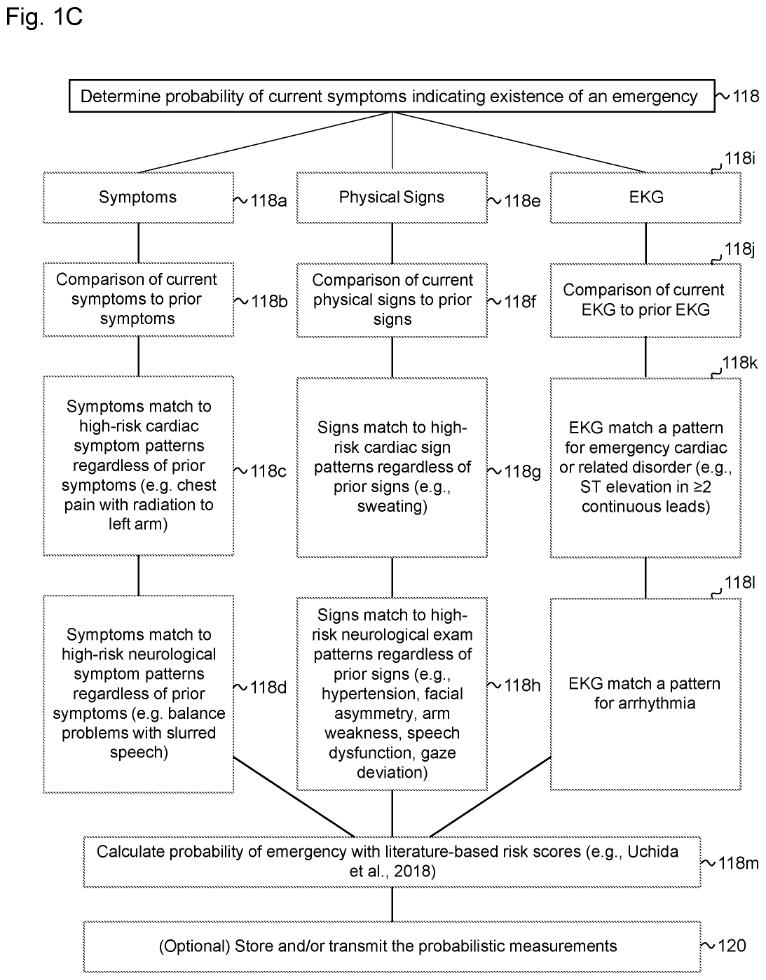

FIG. 1 is a flow diagram that illustrates a method 100 of alerting a user to a likelihood of a health emergency. In optional step 102, a new user is registered. New user registration may include receiving and storing by a processor one or more electronic medical records, or portion thereof, of the user or linking a user's electronic medical records to a user profile for later access. That is, patient history data may be stored locally on a computing device or accessed from a remote database. In optional step 104, a user may input prior cardiac, neurological, or other relevant history for the user, for example related to prior cardiac, neurological or other events or baseline history. In optional step 106, a user may input prior cardiac, neurological, or other physical signs, such as vital signs (e.g., pulse or blood pressure), neurological exam, or electrocardiogram, for example as measured during a routine visit with the user's physician. Optional steps 102-106 may occur at any time and generally will occur when a user is not possibly undergoing a health emergency.

In step 108, a user begins to experience one or more symptoms that may represent a health emergency. In optional step 110, the user may be requested to input (e.g., into a graphical user interface) symptom(s) of concern. The method may then compare those symptom(s) of concern to a list of symptoms correlated with specific health emergencies (e.g., myocardial infarction or stroke) to determine whether further current health data should be obtained and/or compared. For example, a user's symptom may be entirely uncorrelated with a specific health emergency and the method may include notifying (e.g., graphically and/or audibly) the user by a processor that there is no health emergency. In some embodiments, a processor receives a symptom input by the user and determines (e.g., by comparison with a stored list of symptoms correlated with specific health emergencies) that the user should provide additional information upon which it requests, and receives, current health data from the user.

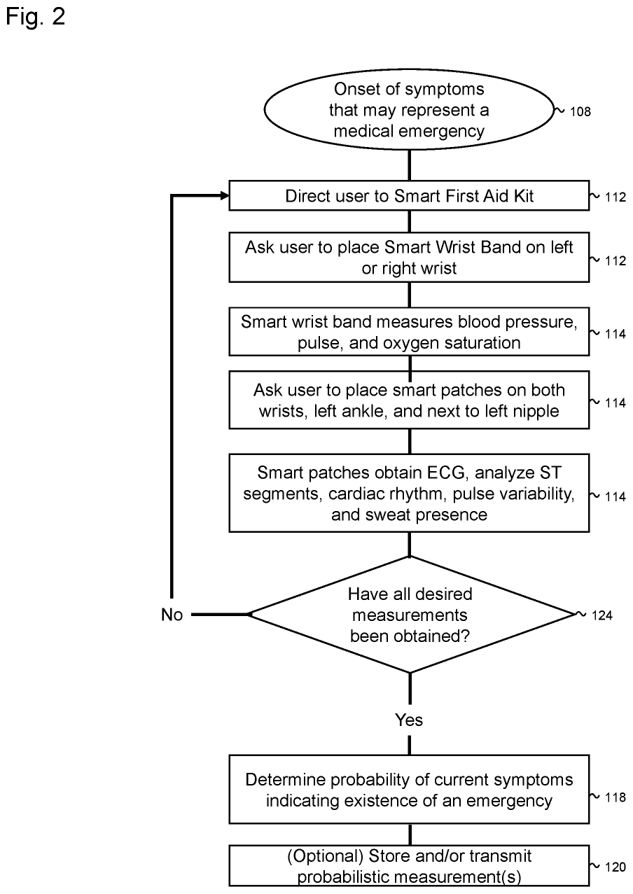

In optional steps 112 and 114, the method may request by a processor (e.g., via a graphical and/or audible notification) that a user acquire current health data, for example using one or more sensors (e.g., as part of a smart first aid kit). The sensor(s) utilized may include one or more of: a 4 lead EKG, an oxygen sensor, a blood pressure monitor (e.g., cuff), a sweat sensor, a temperature probe, and a weight scale. A smart first aid kit may include one or more wearables sized and shaped to be worn by a user and operable to acquire health data and transmit (e.g., wirelessly) the health data to a computing device (e.g., a mobile phone, a smart watch, a tablet, or a personal computer) (e.g., on which method 100 is performed). A smart first aid kit may also include one or more smart patches sized and shaped to be temporarily adhered to skin of a user and operable to acquire health data and transmit (e.g., wirelessly) the health data to a computing device (e.g., a mobile phone, a smart watch, a tablet, or a personal computer) (e.g., on which method 100 is performed). A wearable may include a wrist band or shirt (e.g., with integrated electrode(s) operable to obtain an electrocardiogram). A wrist band may include a blood pressure module, a pulse module, an oxygen saturation module, a sweat monitor module, or a combination thereof. In some embodiments, a smart first aid kit includes two wrist patches, an ankle patch, and a chest (e.g., nipple) patch, for example that are together operable to obtain an electrocardiogram or electrocardiogram data (e.g., a portion of an electrocardiogram) or sweat presence data (e.g., by placing them on both wrists, the left ankle, and the left nipple of a user). In some embodiments, a smart patch is operable to analyze ST segment(s), cardiac rhythm, or pulse variability from the electrocardiogram data or sweat presence from the sweat presence data. In some embodiments, a smart patch is operable to transmit data (e.g., wirelessly) to one or more wearables and the one or more wearable are operable to analyze ST segment(s), cardiac rhythm, or pulse variability from the electrocardiogram data or sweat presence from the sweat presence data. (FIG. 2 illustrates in greater detail a method of using a smart first aid kit to perform method 100. Analogous steps use analogous labels.) A “smart” patch or “smart” wearable (e.g., wrist band) may be smart because, for example, it is operable to automatically sync/transmit data (e.g., wirelessly, such as with Bluetooth®) measured from one or more sensors included therein and/or process such data into a usable form prior to transmission.

In some embodiments, a user manually inputs current health data from non-smart sensor(s), such as a conventional scale, blood pressure cuff, or without use of a sensor (e.g., manually determining a current pulse). Which current health data is requested from a user may be determined based on symptom(s) input by the user in step 110 and/or based on known patient history data for the user.

In step 116, current health data received from the user is compared to patient history data. FIG. 1B shows a detailed view of subroutines that may be used to make comparisons between current health data and patient history data, specifically for potential cardiac and neurological emergencies.

Referring to FIG. 1B, steps 116a-116g may be used if a user is possibly undergoing a cardiac health emergency. In step 116a, a processor determines that patient symptom(s) received from user input match (e.g., partially) a defined set of cardiac symptoms (e.g., stored in a database, locally or remotely from the computing device used by the user). In step 116b, current health data including one or more of intensity, duration, location, and pattern of one or more current cardiac symptoms (e.g., received from one or more sensors) is compared (e.g., matched, at least partially) to patient history data including one or more of intensity, duration, location, and pattern of one or more prior cardiac symptoms or baselines (e.g., one or more of intensity, duration, location, and pattern of the history data and current data are respectively compared). In step 116c, if the comparison determines that the current cardiac symptom(s) are the same or worse than prior cardiac symptom(s) or baseline(s) then one or more probabilistic measurements can be incremented. In step 116d, one or more current physical signs (e.g., blood pressure and/or sweat signs, such as presence or rate) in the current health data are compared to one or more baseline physical signs included in the patient history data. In step 116e, if a processor determines that there is a new abnormality and/or a persistently abnormal physical sign (e.g., over a period of time, such as 30 seconds) from the comparison in step 116d, then one or more probabilistic measurements can be incremented. In step 116f, a current electrocardiogram (EKG) in the current health data is compared (e.g., matched, at least partially) to a baseline electrocardiogram in the patient history data. In step 116g, if the electrocardiogram in the current health data shows changes, when compared to an electrocardiogram in the patient history data, that match (e.g., at least partially) a pattern for emergency cardiac or related disorder (e.g., ST segment elevation in at least two contiguous leads), then one or more probabilistic measurements can be incremented. In some embodiments, only one, only two, or all three of the comparisons in steps 116b, 116d, and 116f are performed during step 116. Any of steps 116c, 116e, and 116g (or a combination thereof) may be performed as part of step 118 (discussed subsequently).

Steps 116h-116p may be used if a user is possibly undergoing a neurological health emergency. In step 116h, a processor determines that patient symptom(s) received from user input match (e.g., partially) a defined set of neurological symptoms (e.g., stored in a database, locally or remotely from the computing device used by the user). In step 116i, current health data including one or more of intensity, duration, and location of one or more current neurological symptoms (e.g., received from one or more sensors) is compared (e.g., matched, at least partially) to patient history data including one or more of intensity, duration, and location of one or more prior neurological symptoms or baselines (e.g., one or more of intensity, duration, and location of the history data and current data are respectively compared). In step 116j, if the comparison determines that the current neurological symptom(s) are the same or worse than prior neurological symptom(s) or baseline(s), then one or more probabilistic measurements can be incremented.

Impaired finger tapping is a sign of many strokes. A computing device (e.g., smartphone) may be equipped with a finger tapping analysis capability. Performance can be judged at baseline and during emergency use in right and left hands. Performance metrics that may be used include speed and/or cadence of tapping. For example, a graphical user interface can request that a user tap an icon in the interface as many times as possible with one hand (e.g., one finger on one hand) at a time within a given period for that hand (e.g., of no more than 10 seconds, no more than 20 seconds, or no more than 30 seconds). In one example, the graphical user interface prompts a user to tap as many times as possible with their right hand within 20 s and then as many times as possible with their left hand within 20 s. After completion of tapping in both hands, speed and/or cadence of the tapping may be analyzed, for example by comparison to a previously recorded baseline.

Voice and facial recognition may also be compared to a baseline to determine whether current neurological symptom(s) are the same or worse than prior neurological symptom(s) or baseline(s) to determine whether to increment one or more probabilistic measurements. For example, a graphical user interface may prompt a user to provide voice recording of a predetermined phrase. Comparison of one or more vocal features determined from the recording as compared to a baseline (e.g., prior recording) may be used to determine whether current neurological symptom(s) are the same or worse than prior neurological symptom(s) or baseline(s). Similarly, facial recognition may be carried out by a camera of a computing device (e.g., smartphone) to assess whether current neurological symptom(s) are the same or worse than prior neurological symptom(s) or baseline(s). For example, facial recognition may be used to search for droop or one or more other signs of neurological distress. Artificial intelligence (e.g., machine learning) may be used to analyze facial recognition and/or vocal recognition information.

In some embodiments, the one or more probabilistic measurements are incremented at step 116j only if the user also has a history of transient ischemic attack (e.g., as input (e.g., upon prompting with a graphical user interface) by the user and/or included in patient history data). In step 116k, one or more current physical signs (e.g., blood pressure and/or sweat signs, such as presence or rate) in the current health data are compared to one or more baseline physical signs included in the patient history data. In step 116l, if a processor determines that there is a new abnormality and/or a persistently abnormal physical sign (e.g., over a period of time, such as 30 seconds) from the comparison in step 116k, then one or more probabilistic measurements can be incremented. In step 116m, a current neurological exam in the current health data is compared to a baseline neurological exam in the patient history data. In step 116n, if the current neurological exam shows changes, when compared to the baseline neurological exam, that match a pattern of emergency neurological disorder (e.g., new language difficulty and right-sided weakness), then one or more probabilistic measurements can be incremented. In step 116o, a current electrocardiogram (EKG) in the current health data is compared (e.g., matched, at least partially) to a baseline electrocardiogram in the patient history data. In step 116p, if the electrocardiogram in the current health data, when compared to an electrocardiogram in the patient history data, shows a new arrhythmia, then one or more probabilistic measurements can be incremented. In some embodiments, only one, only two, only three, or all four of the comparisons in steps 116i, 116k, 116m, and 116o are performed during step 116. Any of steps 116j, 1161, 116n, and 116p (or a combination thereof) may be performed as part of step 118 (discussed subsequently).

Referring back to FIG. 1A, in step 118, one or more probabilistic measurements are determined based at least in part on the comparison in step 116 (of current health data to patient history data). FIG. 1C shows detailed view of subroutines that may be used to determine one or more probabilistic measurements from a comparison of current health data to patient history data for a patient that may be undergoing a cardiac and/or neurological health emergency (e.g., as determined by a processor in response to one or more symptoms of concern received from user input in step 110).

Referring to FIG. 1C, steps 118a-118d may be used, in some embodiments in combination with step 118m, if symptom(s) are used as a basis for determining one or more probabilistic measurements (e.g., as part of the comparison in step 116). In step 118a, a symptoms subroutine is started, for example based on one or more symptoms being included in current health data received from input by a user (e.g., in step 110 or step 112). In step 118b, one or more symptoms included in the current health data are compared to one or more symptoms in patient history data for the user. In step 118c, one or more symptoms included in the current health data are compared to one or more high-risk cardiac symptoms (e.g., independent of any patient history data) (e.g., chest pain with radiation to left arm). In step 118d, one or more symptoms included in the current health data are compared to one or more high-risk neurological symptoms (e.g., independent of any patient history data) (e.g., balance problems with slurred speech). One or more probabilistic measurements may be incremented based on matching of symptoms in any one or more of step 118b, step 118c, or step 118d. For example, there may be a separate probabilistic measurement for likelihood of myocardial infarction, for likelihood of another cardiac emergency, for likelihood of stroke, and for likelihood of another neurological emergency. That is, there may be four different probabilistic measurements that each may be incremented (e.g., adding point(s) to) based on the comparisons. In some embodiments, there are two, not four, probabilistic measurements (e.g., one each for all potential cardiac emergencies and all potential neurological emergencies).

A probabilistic measurement for the symptoms subroutine 118a-d may be determined using step 118m where predetermined risk scores for health emergencies (e.g., cardiac and/or neurological risk scores), for example that correlate symptoms in current health data for a user with predetermined (e.g., literature-based) risk scores for those symptoms. In some embodiments, a probabilistic measurement is incremented by an amount corresponding to a correlation between risk or likelihood and particular current health data. For example, where current health data includes a symptom that is highly correlated with a high risk of a health emergency, a probabilistic measurement may be incremented by a large amount relative to a weak correlation or a correlation with a low risk of the health emergency. In some embodiments, patient history data may be used to determine an amount by which a probabilistic measurement is incremented based on a comparison or matching. For example, if a user has a history of a high-risk symptom, a probabilistic measurement may be incremented differently when current health data includes that symptom than if the user did not have such a history.

Steps 118e-118h may be used, in some embodiments in combination with step 118m, if symptom(s) are used as a basis for determining one or more probabilistic measurements (e.g., as part of the comparison in step 116). In step 118e, a physical signs subroutine is started, for example based on one or more physical signs being included in current health data received from input by a user (e.g., in step 112). In step 118f, one or more symptoms included in the current health data are compared to one or more symptoms in patient history data for the user. In step 118g, one or more physical signs included in the current health data are compared to one or more high-risk cardiac physical signs (e.g., independent of any patient history data) (e.g., sweating, such as presence and/or rate). In step 118h, one or more physical signs included in the current health data are compared to one or more high-risk neurological physical signs (e.g., independent of any patient history data) (e.g., hypertension, facial asymmetry, arm weakness, speech dysfunction, gaze deviation). One or more probabilistic measurements may be incremented based on matching of symptoms in any one or more of step 118f, step 118g, or step 118h. A probabilistic measurement for the physical signs subroutine 118e-h may be determined using step 118m where predetermined risk scores for health emergencies (e.g., cardiac and/or neurological risk scores), for example that correlate physical signs in current health data for a user with predetermined (e.g., literature-based) risk scores for those physical signs.

Steps 118i-1181 may be used, in some embodiments in combination with step 118m, if symptom(s) are used as a basis for determining one or more probabilistic measurements (e.g., as part of the comparison in step 116). In step 118i, an electrocardiogram (EKG) subroutine is started, for example based on an electrocardiogram being included in current health data received from input by a user (e.g., in step 112). In step 118j, an electrocardiogram (e.g., portion thereof) included in the current health data is compared to an electrocardiogram in patient history data for the user. In step 118k, an electrocardiogram included in the current health data is compared to an electrocardiogram for one or more emergency cardiac or related disorders (e.g., by comparing patterns thereof, e.g., to determine whether there is ST segment elevation in at least two continuous leads) (e.g., independent of any patient history data). In step 118l, an electrocardiogram included in the current health data is compared to an electrocardiogram showing an arrhythmia (e.g., independent of any patient history data). One or more probabilistic measurements may be incremented based on matching of symptoms in any one or more of step 118j, step 118k, or step 118l. A probabilistic measurement for the electrocardiogram (EKG) subroutine 118i-1 may be determined using step 118m where predetermined risk scores for health emergencies (e.g., cardiac and/or neurological risk scores), for example that correlate physical signs in current health data for a user with predetermined (e.g., literature-based) risk scores for those physical signs.

In some embodiments, only one, only two, or all three of the symptoms subroutine in steps 118a-d, the physical signs subroutine in steps 118e-h, and the electrocardiogram (EKG) subroutine in steps 118i-1181 are performed during step 118. Any of steps 118b, 118f, and 118i (or a combination thereof) may be performed as part of step 116 (discussed previously). That is, step 116 and step 118 may be performed simultaneously or in an alternating fashion, for example with a first subroutine (e.g., for physical signs), involving comparison of a portion of current health data to a portion of patient history data and determination of one probabilistic measurement, being processed by a processor before a second subroutine (e.g., for an electrocardiogram).

Additionally or alternatively, a processor may prompt a user to input, by a graphical user interface, information regarding whether one or more triggers (e.g., triggering events) have recently occurred, for example recently within a period of time (e.g., the last day, last week, or last month). The presence or absence of one or more triggers, either at all or within a certain period of time, may factor into determining a probabilistic measurement. For example, a process may increment a probabilistic measurement more when one or more triggers is present than if one or more triggers are absent. As another example, a processor may weigh certain input(s) into a probabilistic measurement more when one or more triggers are present than if one or more triggers are absent. A graphical user interface may first prompt a user to provide input to respond to one or more generic questions to determine whether one or more triggers might be present before prompting a user to provide input regarding one or more specific questions about specific trigger(s) (e.g., a user may first be asked to provide input as to whether or not any unexpected events have occurred in the user's life before asking about whether specific events have occurred). As one example, users are generally at a higher risk of having a cardiac emergency after experiencing sudden tragedy, such as unexpected death of a loved one. If one or more symptoms are present for a user that has recently experienced such a loss, a probabilistic measurement determined based on the presence of that symptom may be incremented more due to the conditional presence of the trigger. Moreover, those who have recently experienced certain triggers may be expecting to feel abnormally and therefore be less likely to seek emergency assistance on their own than they normally would be absent using systems and methods disclosed herein. Using one or more triggers to determine one or more probabilistic measurements may be useful for both potential cardiac emergencies and neurological emergencies. One or more triggers used for probabilistic measurement(s) for cardiac emergencies may be different from one or more triggers used for probabilistic measurement(s) for neurological emergencies (or not).

In some embodiments, one or more objective criteria are used to assess the likelihood that a user is experiencing a cardiac emergency and/or neurological emergency. For example, it is known that cardiac emergencies are more likely to occur later in the day (e.g., at night) than earlier in the day. A probabilistic measurement may be determined, at least in part, based on one or more objective criteria, such as time of day. In some embodiments, a probabilistic measurement is incremented more when one or more objective criteria are present. For example, a probabilistic measurement incremented due to the presence of one or more symptoms in a user may be incremented more if it is also nighttime.

Referring back to FIG. 1A, in optional step 120, the one or more probabilistic measurements may be stored and/or transmitted (e.g., to the user's electronic medical record and/or to an emergency medical service). In optional step 122, the method may notify, by a processor (e.g., via one or more graphical and/or audible notifications that are output), the user based on the one or more probabilistic measurements exceeding a threshold. In optional step 122, the method may alternatively or additionally notify, by a processor, an emergency medical service that is remote to the user (e.g., 911) (e.g., by automatically calling the emergency medical service) based on the one or more probabilistic measurements exceeding the threshold. The notification may instruct the user to contact an emergency medical service (e.g., if the method is performed on a device without a call function or Internet function) and/or a physician for the user.

In optional step 124, the method may determine whether all desired measurements have been obtained. The notification in step 122 may indicate a value of at least a portion of the one or more probabilistic measurements (e.g., to assist the user and/or emergency medical service in responding to the health emergency). If not, a processor may request (e.g., using one or more graphical and/or audible notifications, e.g., via a graphical user interface) additional current health data be input in order to make one or more desired measurements. The desired measurement(s) determined to be missing may be, for example, one or more probabilistic measurements that had not been determined based on which current health data was received and/or current health data that was expected based on symptom(s) input by a user and not yet received. (FIG. 1A shows iterating after step 122 and restarting at step 110 but iterating may also occur after any of step 118 or step 120 and/or restart at any of step 112, step 114, or step 116, for example.)

A comparison or matching may be performed in any of a number of different ways. In some embodiments, a comparison or matching involves bitwise comparison of data to determine a degree of similarity. In some embodiments, a machine learning or artificial intelligence algorithm is used to compare different data. In some embodiments, data (e.g., an electrocardiogram) is processed (e.g., filtered, truncated) prior to comparison by a processor. In some embodiments, a comparison or matching may include determining if there is an exact equivalence between a portion of current health data and a portion of patient history data.

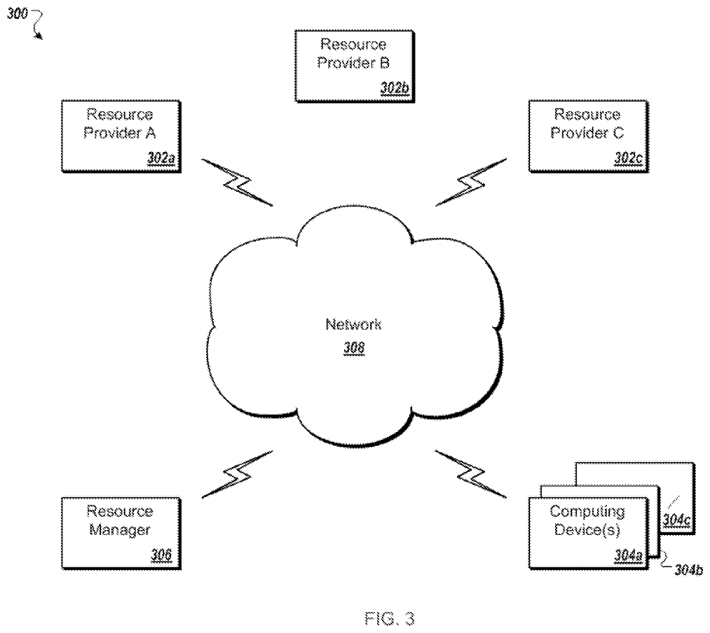

Illustrative embodiments of systems and methods disclosed herein were described above with reference to computations performed locally by a computing device. However, computations performed over a network are also contemplated. FIG. 3 shows an illustrative network environment 300 for use in the methods and systems described herein. In brief overview, referring now to FIG. 3, a block diagram of an illustrative cloud computing environment 300 is shown and described. The cloud computing environment 300 may include one or more resource providers 302a, 302b, 302c (collectively, 302). Each resource provider 302 may include computing resources. In some implementations, computing resources may include any hardware and/or software used to process data. For example, computing resources may include hardware and/or software capable of executing algorithms, computer programs, and/or computer applications. In some implementations, illustrative computing resources may include application servers and/or databases with storage and retrieval capabilities. Each resource provider 302 may be connected to any other resource provider 302 in the cloud computing environment 300. In some implementations, the resource providers 302 may be connected over a computer network 308. Each resource provider 302 may be connected to one or more computing device 304a, 304b, 304c (collectively, 304), over the computer network 308.

The cloud computing environment 300 may include a resource manager 306. The resource manager 306 may be connected to the resource providers 302 and the computing devices 304 over the computer network 308. In some implementations, the resource manager 306 may facilitate the provision of computing resources by one or more resource providers 302 to one or more computing devices 304. The resource manager 306 may receive a request for a computing resource from a particular computing device 304. The resource manager 306 may identify one or more resource providers 302 capable of providing the computing resource requested by the computing device 304. The resource manager 306 may select a resource provider 302 to provide the computing resource. The resource manager 306 may facilitate a connection between the resource provider 302 and a particular computing device 304. In some implementations, the resource manager 306 may establish a connection between a particular resource provider 302 and a particular computing device 304. In some implementations, the resource manager 306 may redirect a particular computing device 304 to a particular resource provider 302 with the requested computing resource.

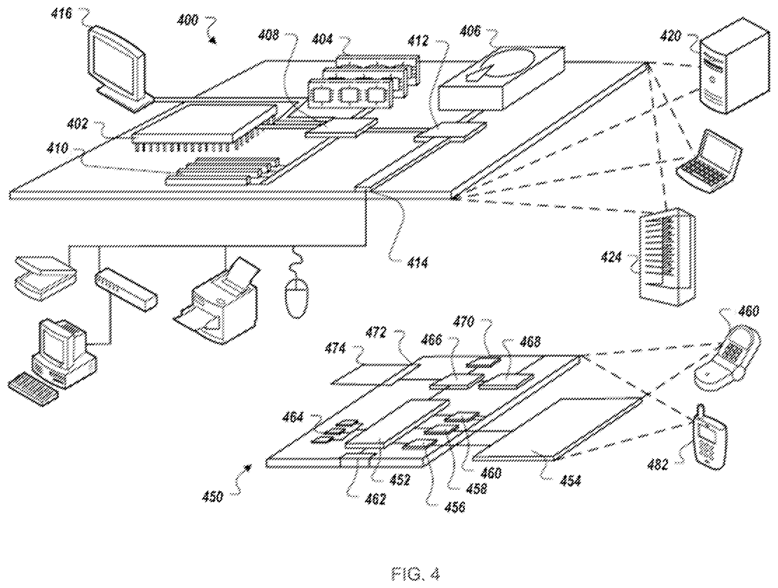

FIG. 4 shows an example of a computing device 400 and a mobile computing device 450 that can be used in the methods and systems described in this disclosure. The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (e.g., one or more processors) of any number of computing devices (e.g., one or more computing devices). Furthermore, where a function is described as being performed by “a processor”, this encompasses embodiments wherein the function is performed by any number of processors (e.g., one or more processors) of any number of computing devices (e.g., one or more computing devices) (e.g., in a distributed computing system).

The memory 404 stores information within the computing device 400. In some implementations, the memory 404 is a volatile memory unit or units. In some implementations, the memory 404 is a non-volatile memory unit or units. The memory 404 may also be another form of computer-readable medium, such as a magnetic or optical disk.

The storage device 406 is capable of providing mass storage for the computing device 400. In some implementations, the storage device 406 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 402), perform one or more methods, such as those described above. The instructions can also be stored by one or more storage devices such as computer- or machine-readable mediums (for example, the memory 404, the storage device 406, or memory on the processor 402).

The high-speed interface 408 manages bandwidth-intensive operations for the computing device 400, while the low-speed interface 412 manages lower bandwidth-intensive operations. Such allocation of functions is an example only. In some implementations, the high-speed interface 408 is coupled to the memory 404, the display 416 (e.g., through a graphics processor or accelerator), and to the high-speed expansion ports 410, which may accept various expansion cards (not shown). In the implementation, the low-speed interface 412 is coupled to the storage device 406 and the low-speed expansion port 414. The low-speed expansion port 414, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 400 may be implemented in a number of different forms, as shown in the figure. For example, it may be implemented as a standard server 420, or multiple times in a group of such servers. In addition, it may be implemented in a personal computer such as a laptop computer 422. It may also be implemented as part of a rack server system 424. Alternatively, components from the computing device 400 may be combined with other components in a mobile device (not shown), such as a mobile computing device 450. Each of such devices may contain one or more of the computing device 400 and the mobile computing device 450, and an entire system may be made up of multiple computing devices communicating with each other.

The mobile computing device 450 includes a processor 452, a memory 464, an input/output device such as a display 454, a communication interface 466, and a transceiver 468, among other components. The mobile computing device 450 may also be provided with a storage device, such as a micro-drive or other device, to provide additional storage. Each of the processor 452, the memory 464, the display 454, the communication interface 466, and the transceiver 468, are interconnected using various buses, and several of the components may be mounted on a common motherboard or in other manners as appropriate.

The processor 452 can execute instructions within the mobile computing device 450, including instructions stored in the memory 464. The processor 452 may be implemented as a chipset of chips that include separate and multiple analog and digital processors. The processor 452 may provide, for example, for coordination of the other components of the mobile computing device 450, such as control of user interfaces, applications run by the mobile computing device 450, and wireless communication by the mobile computing device 450.

The processor 452 may communicate with a user through a control interface 458 and a display interface 456 coupled to the display 454. The display 454 may be, for example, a TFT (Thin-Film-Transistor Liquid Crystal Display) display or an OLED (Organic Light Emitting Diode) display, or other appropriate display technology. The display interface 456 may comprise appropriate circuitry for driving the display 454 to present graphical and other information to a user. The control interface 458 may receive commands from a user and convert them for submission to the processor 452. In addition, an external interface 462 may provide communication with the processor 452, so as to enable near area communication of the mobile computing device 450 with other devices. The external interface 462 may provide, for example, for wired communication in some implementations, or for wireless communication in other implementations, and multiple interfaces may also be used.

The memory 464 stores information within the mobile computing device 450. The memory 464 can be implemented as one or more of a computer-readable medium or media, a volatile memory unit or units, or a non-volatile memory unit or units. An expansion memory 474 may also be provided and connected to the mobile computing device 450 through an expansion interface 472, which may include, for example, a SIMM (Single In Line Memory Module) card interface. The expansion memory 474 may provide extra storage space for the mobile computing device 450, or may also store applications or other information for the mobile computing device 450. Specifically, the expansion memory 474 may include instructions to carry out or supplement the processes described above, and may include secure information also. Thus, for example, the expansion memory 474 may be provided as a security module for the mobile computing device 450, and may be programmed with instructions that permit secure use of the mobile computing device 450. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 452), perform one or more methods, such as those described above. The instructions can also be stored by one or more storage devices, such as one or more computer- or machine-readable mediums (for example, the memory 464, the expansion memory 474, or memory on the processor 452). In some implementations, the instructions can be received in a propagated signal, for example, over the transceiver 468 or the external interface 462.

The mobile computing device 450 may communicate wirelessly through the communication interface 466, which may include digital signal processing circuitry where necessary. The communication interface 466 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 468 using a radio-frequency. In addition, short-range communication may occur, such as using a Bluetooth®, Wi-Fi™, or other such transceiver (not shown). In addition, a GPS (Global Positioning System) receiver module 470 may provide additional navigation- and location-related wireless data to the mobile computing device 450, which may be used as appropriate by applications running on the mobile computing device 450.

The mobile computing device 450 may also communicate audibly using an audio codec 460, which may receive spoken information from a user and convert it to usable digital information. The audio codec 460 may likewise generate audible sound for a user, such as through a speaker, e.g., in a handset of the mobile computing device 450. Such sound may include sound from voice telephone calls, may include recorded sound (e.g., voice messages, music files, etc.) and may also include sound generated by applications operating on the mobile computing device 450.

The mobile computing device 450 may be implemented in a number of different forms, as shown in the figure. For example, it may be implemented as a cellular telephone 480. It may also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

What is claimed is:

1. A method for alerting a user to a likelihood of a health emergency for the user, the method comprising:
receiving, by a processor of a computing device (e.g., a mobile phone, smart watch, tablet, or personal computer), patient history data for the user;
receiving, by the processor, current health data from the user; and
determining, by the processor, one or more probabilistic measurements at least in part by comparing the current health data to the patient history data, the one or more probabilistic measurements corresponding to the likelihood of a health emergency (e.g., a cardiac emergency, e.g., heart attack, and/or neurological emergency, e.g., stroke).

2. The method of claim 1, comprising:
determining, by the processor, that at least a portion of the one or more probabilistic measurements exceed a threshold; and
outputting, by the processor, a notification (e.g., a graphical notification, an auditory notification, or both) to the user (e.g., wherein the notification indicates a value of the at least a portion of the one or more probabilistic measurements) (e.g., wherein the notification indicates a likelihood of a health emergency and/or instructs the user to contact an emergency service provider).

3. The method of claim 1 or claim 2, comprising:
determining, by the processor, that at least a portion of the one or more probabilistic measurements exceed a threshold; and
automatically notifying (e.g., calling), by the processor, a remote emergency service provider to inform the provider of a medical emergency for the user (e.g., automatically calling 911).

4. The method of claim 3, comprising transmitting, by the processor, to the remote emergency service provider, at least a portion of the current health data and/or at least a portion of the one or more probabilistic measurements.

5. The method of any one of claims 1-4, wherein receiving the patient history data comprises receiving, by the processor, at least a portion of the patient history data from input provided by the user (e.g., into a graphical user interface).

6. The method of any one of claims 1-5, wherein receiving the patient history data comprises receiving, by the processor, at least a portion of the patient history data from an electronic medical records database.

7. The method of any one of claims 1-6, wherein receiving the current health data comprises receiving, by the processor, input data from one or more sensors (e.g., integrated into one or more wearables and/or one or more smart patches) in communication with the processor.

8. The method of any one of claims 1-7, wherein receiving the current health data comprises receiving, by the processor, at least a portion of the current health data from input manually provided by the user (e.g., into a graphical user interface).

9. The method of any one of claims 1-8, wherein the patient history data and the current health data both comprise data corresponding to one or more vital signs, neurological status, an electrocardiogram, or one or more other cardiovascular parameters.

10. The method of any one of claims 1-9, comprising:
determining, by the processor, that one or more probabilistic measurements have not been made within a period of time; and
requesting, with a graphical user interface, by the processor, that the user input additional current health data.

11. The method of claim 10, comprising receiving, by the processor, the additional current health data (e.g., corresponding to one or more vital signs, neurological status, an electrocardiogram, or one or more other cardiovascular parameters) from one or more sensors (e.g., integrated into one or more wearables and/or one or more smart patches) in communication with the processor and/or input manually provided by the user (e.g., into a graphical user interface).

12. The method of any one of claims 1-11, comprising:
receiving, by the processor, input from the user of one or more symptoms of concern;
determining, by the processor, the one or more symptoms are indicative of a potential health emergency; and
requesting, by the processor, (e.g., by a graphical user interface) the current health data based on the input corresponding to the one or more symptoms of concern.

13. The method of claim 12, wherein requesting the current health data comprises providing, by the processor, a notification to the user to request the user use one or more sensors to acquire at least a portion of the current health data.

14. The method of any one of claims 1-13, wherein the health emergency is a cardiac event (e.g., heart attack) and/or neurological event (e.g., stroke), and/or other related medical condition (e.g., pulmonary embolism).

15. The method of any one of claims 1-14, wherein the patient history data comprise one or more of intensity, duration, location, and pattern of one or more prior cardiac symptoms (e.g., chest pain) or baselines and the current health data comprise one or more of intensity, duration, and location of one or more current cardiac symptoms and comparing the current health data to the patient history data comprises:
comparing intensity of the one or more current cardiac symptoms to intensity of the one or more prior cardiac symptoms or baselines,
comparing duration of the one or more current cardiac symptoms to duration of the one or more prior cardiac symptoms or baselines,
comparing location of the one or more current cardiac symptoms to location of the one or more prior cardiac symptoms or baselines,
comparing pattern of the one or more current cardiac symptoms to pattern of the one or more prior cardiac symptoms or baselines, or
a combination thereof.

16. The method of claim 15, comprising:
receiving, by the processor, input from the user of one or more symptoms of concern;
matching, by the processor, the one or more symptoms of concern to one or more symptoms of a cardiac emergency; and
requesting, by the processor, (e.g., by a graphical user interface) the current health data based on the matching.

17. The method of claim 15 or claim 16, comprising:
determining, by the processor, that the current health data corresponds to a cardiac health status no better than a cardiac health status that the patient history data corresponds to; and
in response, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

18. The method of any one of claims 1-17, wherein the patient history data comprise one or more baseline cardiac physical signs (e.g., blood pressure, sweat (e.g., rate and/or composition thereof)) and the current health data comprise one or more current cardiac physical signs (e.g., blood pressure, sweat (e.g., rate and/or composition thereof)) (e.g., received from one or more sensors in communication with the processor) and comparing the current health data to the patient history data comprises comparing the one or more current cardiac physical signs to the one or more baseline cardiac physical signs.

19. The method of claim 18, comprising receiving, by the processor, the one or more current physical signs from one or more sensors in communication with the processor.

20. The method of claim 18 or claim 19, wherein:
comparing the one or more current cardiac physical signs to the one or more cardiac baseline physical signs comprises determining, by the processor, a presence of (i) a new abnormality in the current health data, (ii) a persistent abnormal physical sign in the current health data, or (iii) both (i) and (ii), and
the method comprises, in response to determining the presence of (i), (ii), or (iii), altering, by the processor, one or more of the one or more probabilistic measurements (e.g., incrementing the one or more of the one or more probabilistic measurements, e.g., by increasing a value of (e.g., adding points to) one or more of the one or more probabilistic measures).

21. The method of claim 20, comprising determining, by the processor, the presence of (ii) or (iii) and, in response, requesting, by the processor, additional current health data comprising one or more of the one or more current cardiac physical signs.

22. The method of any one of claims 1-21, wherein the current health data comprise at least a portion of a current electrocardiogram (e.g., received from one or more sensors in communication with the processor) and the patient history data comprise at least a portion of a baseline electrocardiogram and comparing the current health data to the patient history data comprises comparing the at least a portion of the current electrocardiogram to the at least a portion of the baseline electrocardiogram.

23. The method of claim 22, comprising:
determining, by the processor, that the at least a portion of the current electrocardiogram compared to the at least a portion of the baseline electrocardiogram matches a pattern for a cardiac emergency (e.g., ST segment elevation in at least two contiguous leads); and
in response, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

24. The method of any one of claims 1-23, wherein determining the one or more probabilistic measurements comprises totaling one or more scores indicative of a cardiac emergency based on comparing one or more cardiac symptoms, one or more physical signs, an electrocardiogram, or combinations thereof in the current health data to corresponding one(s) in the patient history data.

25. The method of claim 24, comprising:
determining, by the processor, that the one or more total scores exceed one or more thresholds; and
automatically notifying, by the processor, the user (e.g., graphically and/or audibly) and/or a remote emergency service provider.

26. The method of any one of claims 1-25, wherein the patient history data comprise one or more of intensity, duration, and location of one or more prior neurological symptoms (e.g., slurred speech, balance problems, right-sided weakness) or baselines and the current health data comprise one or more of intensity, duration, and location of one or more current neurological symptoms and comparing the current health data to the patient history data comprises:
comparing intensity of the one or more current neurological symptoms to intensity of the one or more prior neurological symptoms or baselines,
comparing duration of the one or more current neurological symptoms to duration of the one or more prior neurological symptoms or baselines,
comparing location of the one or more current neurological symptoms to location of the one or more prior neurological symptoms or baselines, or
a combination thereof.

27. The method of claim 26, comprising:
receiving, by the processor, input from the user of one or more symptoms of concern;
matching, by the processor, the one or more symptoms of concern to one or more symptoms of a neurological emergency; and
requesting, by the processor, (e.g., by a graphical user interface) the current health data based on the matching.

28. The method of claim 26 or claim 27, comprising:
determining, by the processor, that the current health data corresponds to a neurological health status no better than a neurological health status that the patient history data corresponds to; and
in response, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

29. The method of any one of claims 1-28, wherein the patient history data comprise one or more baseline neurological physical signs (e.g., blood pressure, sweat (e.g., rate and/or composition thereof)) and the current health data comprise one or more current neurological physical signs (e.g., blood pressure, sweat (e.g., rate and/or composition thereof)) (e.g., received from one or more sensors in communication with the processor) and comparing the current health data to the patient history data comprises comparing the one or more current neurological physical signs to the one or more baseline neurological physical signs.

30. The method of claim 29, comprising receiving, by the processor, the one or more current neurological physical signs from one or more sensors in communication with the processor.

31. The method of claim 29 or claim 30, wherein:
comparing the one or more current neurological physical signs to the one or more baseline neurological physical signs comprises determining, by the processor, a presence of (i) a new abnormality in the current health data, (ii) a persistent abnormal physical sign in the current health data, or (iii) both (i) and (ii), and
the method comprises, in response to determining the presence of (i), (ii), or (iii), altering, by the processor, one or more of the one or more probabilistic measurements (e.g., incrementing the one or more of the one or more probabilistic measurements, e.g., by increasing a value of (e.g., adding points to) one or more of the one or more probabilistic measures).

32. The method of claim 31, comprising determining, by the processor, the presence of (ii) or (iii) and, in response, requesting, by the processor, additional current health data comprising one or more of the one or more current neurological physical signs.

33. The method of any one of claims 1-32, wherein the current health data comprise at least a portion of a current electrocardiogram (e.g., received from one or more sensors in communication with the processor) and the patient history data comprise at least a portion of a baseline electrocardiogram and comparing the current health data to the patient history data comprises comparing the at least a portion of the current electrocardiogram to the at least a portion of the baseline electrocardiogram.

34. The method of claim 33, comprising:
determining, by the processor, that the at least a portion of the current electrocardiogram comprises a new arrhythmia compared to the at least a portion of the baseline electrocardiogram; and
in response, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

35. The method of any one of claims 1-34, wherein the patient history data comprise a current neurological exam and the current health data comprise a baseline neurological exam and comparing the current health data to the patient history data comprises comparing the current neurological exam to the baseline neurological exam.

36. The method of claim 35, wherein:
comparing the current neurological exam to the baseline neurological exam comprises determining, by the processor, the current neurological exam comprises a change from the baseline neurological exam that matches a neurological emergency (e.g., new language difficulty and right-sided weakness), and
the method comprises, in response to determining the change that matches the neurological emergency, altering, by the processor, one or more of the one or more probabilistic measurements (e.g., incrementing the one or more of the one or more probabilistic measurements, e.g., by increasing a value of (e.g., adding points to) one or more of the one or more probabilistic measures).

37. The method of any one of claims 1-36, wherein determining the one or more probabilistic measurements comprises totaling one or more scores indicative of a neurological emergency based on comparing one or more neurological symptoms, one or more physical signs, an electrocardiogram, or combinations thereof in the current health data to corresponding one(s) in the patient history data.

38. The method of claim 37, comprising:
determining, by the processor, that the one or more total scores exceed one or more thresholds; and
automatically notifying, by the processor, the user (e.g., graphically and/or audibly) and/or a remote emergency service provider.

39. The method of any one of claims 1-38, further comprising determining, by the processor, the one or more probabilistic measurements at least in part by matching the current health data to one or more independent baseline criteria.

40. The method of claim 39, wherein the one or more independent baseline criteria comprise one or more high-risk symptoms, one or more high-risk physical signs, or a high-risk electrocardiogram.

41. The method of claim 39 or claim 40, comprising altering, by the processor, the one or more probabilistic measurements based on the matching to the one or more independent baseline criteria independent of the patient history data.

42. The method of any one of claims 1-41, wherein the one or more probabilistic measurements are determined using predetermined risk scores for health emergencies (e.g., cardiac and/or neurological risk scores).

43. The method of any one of claims 1-42, wherein the patient history data comprise one or more baseline physical signs (e.g., blood pressure, sweat (e.g., rate and/or composition thereof)) and the current health data comprise one or more current physical signs (e.g., blood pressure, sweat (e.g., rate and/or composition thereof)) (e.g., received from one or more sensors in communication with the processor) and comparing the current health data to the patient history data comprises comparing the one or more current physical signs to the one or more baseline physical signs.

44. The method of claim 43, comprising receiving, by the processor, the one or more current physical signs from one or more sensors in communication with the processor.

45. The method of claim 43 or claim 44, wherein comparing the one or more current physical signs to the one or more baseline physical signs comprises determining, by the processor, a presence of (i) a new abnormality in the current health data, (ii) a persistent abnormal physical sign in the current health data, or (iii) both (i) and (ii) and the method comprises, in response to determining the presence of (i), (ii), or (iii), altering, by the processor, one or more of the one or more probabilistic measurements (e.g., incrementing the one or more of the one or more probabilistic measurements, e.g. by increasing a value of (e.g., adding points to) one or more of the one or more probabilistic measures).

46. The method of any one of claims 1-45, comprising:
receiving, by the processor, input corresponding to one or more triggers and/or one or more objective criteria;
determining, by the processor, from the input that the one or more triggers and/or the one or more objective criteria are present (e.g., wherein the input corresponds to one or more affirmative responses);
determining, by the processor, the one or more probabilistic measurements based, at least in part, on the input corresponding to the one or more triggers and/or one or more objective criteria (e.g., incrementing one or more of the one or more probabilistic measurements more due to the input).

47. The method of any one of claims 1-46, comprising:
receiving, by the processor, input from the user of one or more symptoms of concern;
determining, by the processor, that the current health data (and optionally the patient history data) is incomplete for the one or more symptoms of concern.

48. The method of claim 47, comprising, upon determining that the current health data is incomplete, requesting, by the processor, (e.g., by a graphical user interface) additional current health data.

49. The method of claim 47 or claim 48, comprising receiving, by the processor, the additional current health data, wherein the additional current health data corresponds to a different type of measurement than current health data.

50. The method of claim 49, wherein the additional current health data is from one or more second sensors and the current health data is from one or more first sensors that are different from the one or more second sensors (e.g., in one or more different wearables and/or one or more different smart patches).

51. The method of claim 50, wherein the additional current health data is received from one or more smart patches and the current health data or is received from one or more wearables or wherein the additional current health data is received from one or more wearables and the current health data or is received from one or more smart patches.

52. A system comprising a processor and a memory, the memory having instructions stored thereon that, when executed by the processor, cause the processor to perform the method of any one of claims 1-51.

53. The system of claim 52, wherein the processor and the memory are comprised in a mobile phone, smart watch, tablet, or personal computer.

54. A smart first aid kit for acquiring health data from a user when the user may be undergoing a health emergency, the kit comprising:
one or more wearables sized and shaped to be worn by the user and operable to acquire first health data and transmit (e.g., wirelessly) the first health data to a computing device (e.g., a mobile phone, a smart watch, a tablet, or a personal computer); and
one or more smart patches sized and shaped to be temporarily adhered to skin of the user and operable to acquire second health data and transmit (e.g., wirelessly) the second health data to a computing device (e.g., mobile phone, smart watch, tablet, or personal computer).

55. The smart first aid kit of claim 54, wherein the one or more wearables comprises a wrist band, a shirt (e.g., with integrated electrodes), or both.

56. The smart first aid kit of claim 55, wherein the one or more wearables comprises the wrist band and the wrist band comprises a blood pressure module, a pulse module, an oxygen saturation module, a sweat monitor module (e.g., a sweat rate monitor module), or a combination thereof.

57. The smart first aid kit of any one of claims 54-56, wherein the one or more smart patches comprises two wrist patches, an ankle patch, and a chest (e.g., nipple) patch.

58. The smart first aid kit of any one of claims 54-57, wherein the one or more smart patches comprises a plurality of smart patches that are together operable to acquire electrocardiogram data or sweat presence data.

59. The smart first aid kit of claim 58, wherein (i) the one or more smart patches are operable to analyze ST segment(s), cardiac rhythm, or pulse variability from the electrocardiogram data or sweat presence from the sweat presence data or (ii) the one or more smart patches are operable to transmit data (e.g., wirelessly) to the one or more wearables and the one or more wearable are operable to analyze ST segment(s), cardiac rhythm, or pulse variability from the electrocardiogram data or sweat presence from the sweat presence data.

60. A method for acquiring health data from a user when the user may be undergoing a health emergency, the method comprising:
providing, by a processor of a computing device (e.g., a mobile phone, a smart watch, a tablet, or a personal computer), a notification (e.g., graphically or audibly) to acquire health data using one or more smart patches; and
receiving, by the processor, health data from a plurality of smart patches that have been placed at both wrists, the left ankle, and the left nipple of the user, wherein the health data is electrocardiogram data or sweat presence data.
